(19) United States
(12) Patent Application Publication
(10) Pub. No.: US 2012/0231542 A1
(43) Pub. Date: Sep. 13, 2012

(54) Biologically Active Human Umbilical Cord Blood Cell Extract Compounds and Methods

(75) Inventors: Erik J. Woods, Indianapolis, IN (US); Sreedhar Thirumala, Indianapolis, IN (US); Dan Zhou, Avon, IN (US); W. Scott Goebel, Indianapolis, IN (US)

(73) Assignee: GENERAL BIOTECHNOLOGY, LLC, Indianapolis, IN (US)

(21) Appl. No.: 13/417,890

(22) Filed: Mar. 12, 2012

Related U.S. Application Data
(60) Provisional application No. 61/451,813, filed on Mar. 11, 2011.

(51) Int. Cl. C12N 5/0775; C12N 5/071

(52) U.S. Cl. 435/366; 435/408

(57) ABSTRACT

Compositions and methods for culturing therapeutic cells are provided herein. According to at least one embodiment, compositions comprising cord blood plasma and lysed platelets and methods for making and using same are provided herein.

### PRIORITY

The present application claims priority to U.S. Provisional Patent Application Ser. No. 61/451,813, filed Mar. 11, 2011, the entirety of which is incorporated herein by reference.

### BACKGROUND

Mesenchymal stem cells (MSCs) are emerging as a promising cell therapy concept for a wide range of tissue types. MSCs can be induced to proliferate within damaged tissues and organs and improve regeneration after ischemic, metabolic and toxic organ injuries. MSCs can be readily isolated and expanded into large numbers ex vivo. Fetal Calf Serum (FCS) or Fetal Bovine Serum (FBS) has been widely used to culture the MSCs since this type of serum lacks high concentrations of components which inhibit cell growth and instead contain growth factors that support cell growth. However, culture of cells in FBS exposes the patient to xenogeneic bovine proteins or antigens which may elicit an immunologic response, and potentially exposes the patient to infectious risks such as bovine spongiform encephalopathy (BSE; aka “mad cow disease). Further, FBS cannot be effectively used for cell proliferation of most stem cells at reduced concentrations.

A variety of human supplements have been postulated as alternatives to FBS/FCS to provide nutrients, attachment factors and growth factors, such as human serum, plasma and platelet derivatives such as platelet lysate and platelet released factors. Blood serum, the liquid fraction of blood that remains after clotting, has been used as a major component of tissue culture media. Blood plasma, the liquid fraction of blood that has not been allowed to clot, is less effective than serum for various tissue culture lines.

Generation of adult human platelet lysate (hPL) from apheresis units of human blood platelets has been reported as one suitable alternative to FBS for culture of human MSC. Platelet units from which hPL is generated are generally intended to be used for transfusion into bleeding or thrombocytopenic patients, and as such are appropriately screened by blood banks for infectious agents. However, generating hPL from platelets by apheresis is expensive and particularly cost-prohibitive for high volume generation of culture media, since it is intended for a transfusion product and not for the manufacture of a serum substitute for cell culture. Further, attempts to make adult hPL more cost effective through reducing the concentration of the adult hPL used in culturing MSCs have indicated that the efficacy of such a solution declines significantly as the concentration is reduced.

As such, a cost effective composition for culturing MSCs and other stem cells would be greatly appreciated, particularly a composition displaying an ability to.

### SUMMARY

According to certain embodiments, a composition for culturing therapeutic cells comprises human platelet lysate and plasma derived from cord blood, wherein the concentration of platelet lysate is approximately the equivalent of 1×10⁹ platelets/ml of composition, and wherein the plasma and/or platelets are operable to culture one or more therapeutic cells. In certain optional embodiments, the composition of, further comprises approximately 0.5 mM/mL to about 5 mM/mL calcium chloride. According to at least one embodiment, the the concentration of platelet lysate is approximately the equivalent of 0.5×10⁹ platelets/ml of composition. According to at least one additional embodiment, the concentration of platelet lysate is approximately the equivalent of 0.5×10⁹ platelets/ml of composition.

According to certain embodiments, the composition is operable to proliferate human mesenchymal stem cells or dental papillary stem cells. According to other embodiments, the composition is operable to proliferate human stem cells at a rate less than double that of fetal bovine serum under identical conditions. According to certain embodiments, the composition further comprises heparin.

According to at least one embodiment, a method for culturing therapeutic cells comprises the steps of providing a therapeutic cell; providing a composition derived from cord blood consisting essentially of combined human platelet lysate and plasma, wherein the concentration of platelet lysate is approximately the equivalent of 1×10⁹ platelets/ml of composition; and culturing the therapeutic cell on the composition. According to certain optional embodiments, the provided composition further comprises approximately 0.5 mM/mL to about 5 mM/mL calcium chloride.

According to certain optional embodiments, the concentration of platelet lysate is approximately the equivalent of 0.5×109 platelets/ml of composition. In at least one optional embodiment, the concentration of platelet lysate is approximately the equivalent of 0.5×10⁹ platelets/ml of composition. According to certain embodiments, the therapeutic cell is mesenchymal stem cells or dental papillary stem cells.

According to at least one embodiment, a composition for culturing therapeutic cells consists essentially of human platelet lysate and plasma derived from cord blood, wherein the concentration of platelet lysate is approximately the equivalent of 1×10⁹ platelets/ml of composition, and wherein the plasma and/or platelets are operable to culture one or more therapeutic cells.

### BRIEF DESCRIPTION

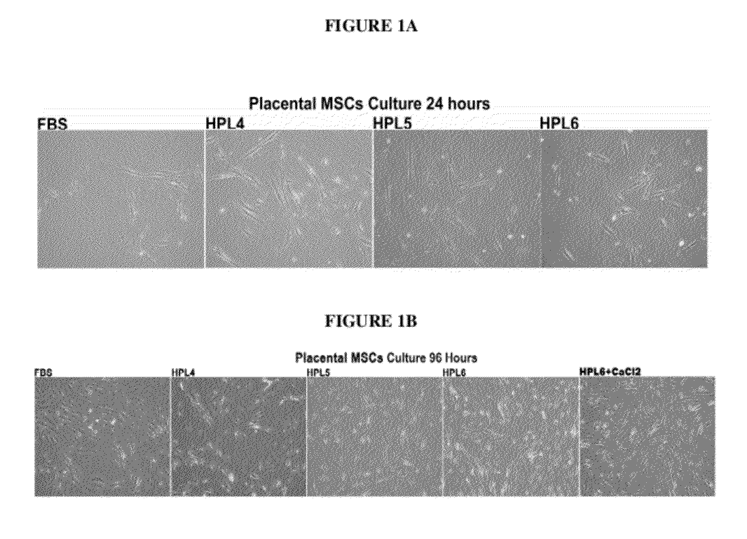

FIG. 1A shows a series of images displaying the growth of placental mesenchymal stem cells cultured in vitro for 24 hours cultured on fetal bovine serum versus various mixtures of human platelet lysate according to at least one embodiment herein.

FIG. 1B shows a series of images displaying the growth of placental mesenchymal stem cells cultured in vitro for 96 hours cultured on fetal bovine serum versus various mixtures of human platelet lysate according to at least one embodiment herein.

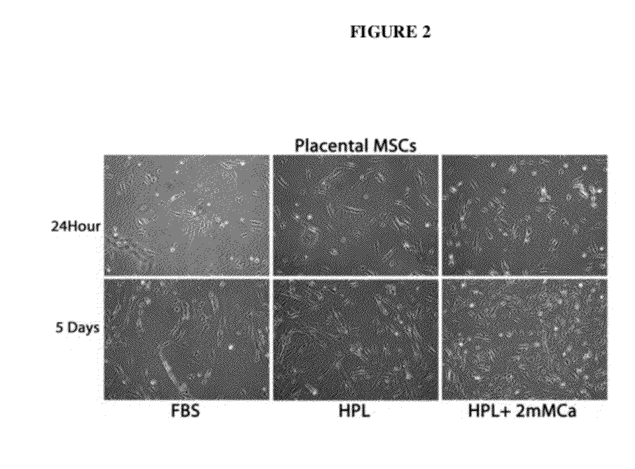

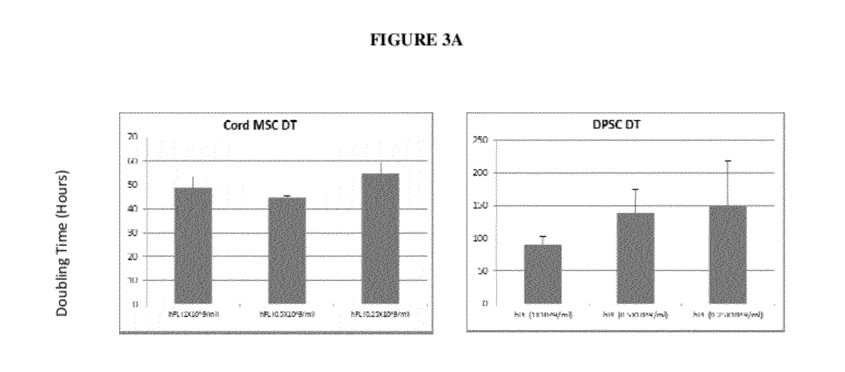

FIG. 2 shows a series of slides displaying the morphology of placental mesenchymal stem cells cultured in vitro for 24 hours and 5 days, as cultured on fetal bovine serum as compared to mixtures of human platelet lysate compositions and human platelet lysate compositions including CaCl₂ according to at least one embodiment herein.

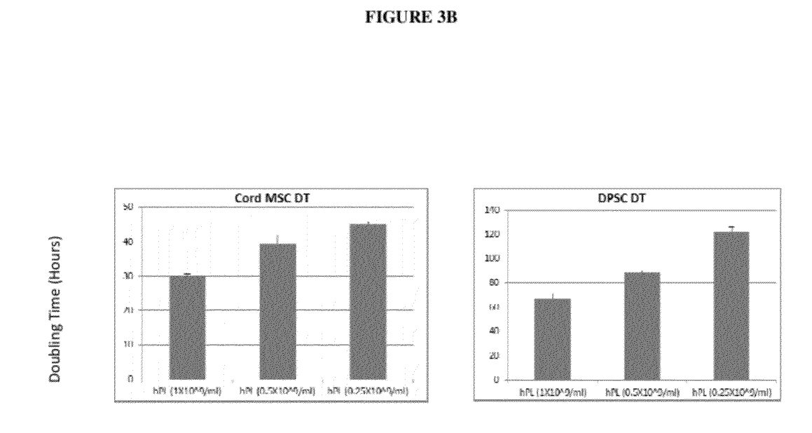

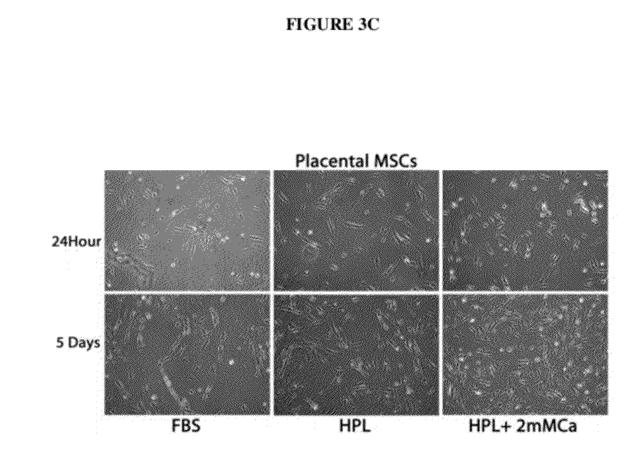

FIGS. 3A-3B are a bar graphs displaying the doubling time of stem cells when cultured in vitro on fetal bovine serum versus various mixtures of human platelet lysate according to at least one embodiment herein.

FIG. 3C is a series of slides displaying the morphology of stem cells cultured in the compositions disclosed in FIGS. 3A-3B.

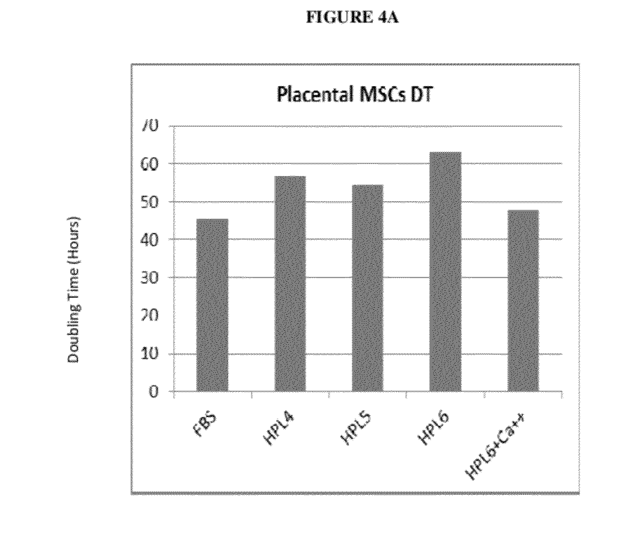

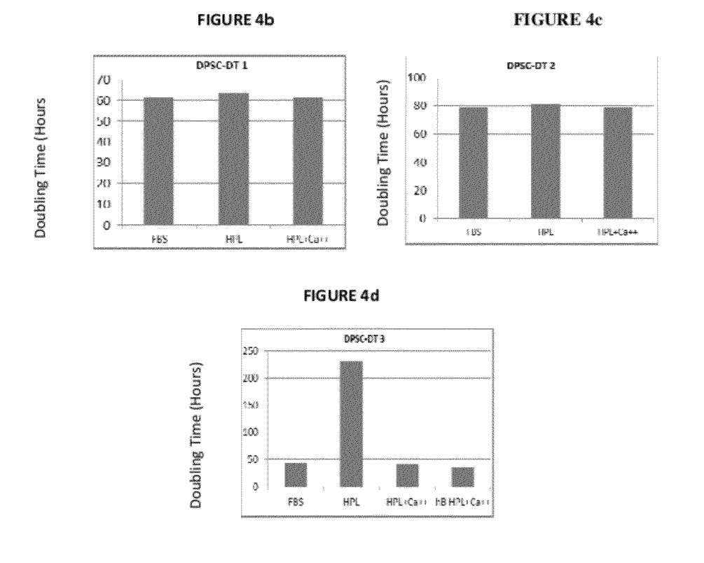

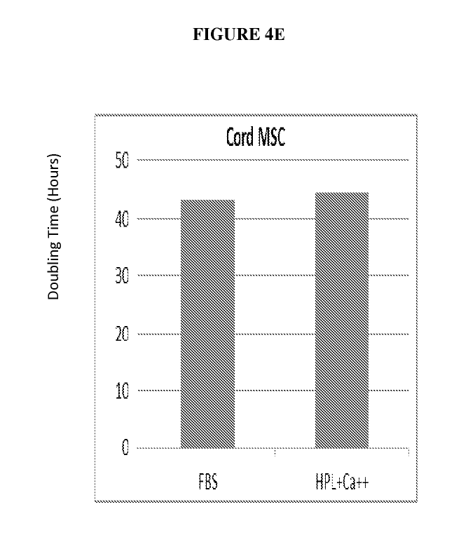

FIG. 4E is a bar graph displaying the doubling time of identified stem cells when cultured in vitro on fetal bovine serum versus various mixtures of human platelet lysate according to at least one embodiment herein.

### DETAILED DESCRIPTION

For the purposes of promoting an understanding of the principles of the invention, reference will now be made to the embodiments described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

Umbilical cord blood is a rich source of hematopoietic stem cells (HSC) for transplantation. In cord blood banks, HSC from donated cord blood units are cryopreserved for future hematopoietic transplantation. However, only 10-20% of donated cord blood units are suitable for cryopreservation for future transplantation. The remaining 80-90% of donated cord blood units is typically discarded.

According to certain embodiments herein, it has been found that compositions comprising human platelet lysate (hPL) derived from from cord blood (CB) and/or related tissue is suitable for the culture of human stem cells, including MSCs. While cord blood units are of much smaller volume than apheresis platelet units, many dozens to hundreds of non-transplantable cord blood units will become available in a cord blood bank over the course of a year, permitting the manufacture of relatively large volumes of hPL for cell culture. Significantly, cord blood hPL is a human product which can be screened for infectious diseases similar to other human blood products, making it a very suitable FBS substitute for human cell manufacturing.

The present invention contemplates a composition for culturing stem cells, the composition including human platelet lysate (hPL) derived from human umbilical cord blood (CB) and a method for preparing the composition. According to at least one embodiment, The hPL is treated as follows:
Approximately one CB unit is centrifuged in 50 mL conical tubes at 1850 rpm for 15 minutes to collect the plasma. Optionally, the plasma is transferred to new tubes, and the new tubes filled with plasma are centrifuged at 1340 rpm for 10 minutes to substantially remove the remaining leukocytes and erythrocytes. Optionally, the plasma is inserted into a large sterile bottle and mix with plasma obtained from about additional CB units treated similarly to the above. According to at least one embodiment, approximately 10-20 CB plasma units are combined in the bottle. Thereafter, the platelets in the plasma are counted and the total volume of the plasma is determined to obtain the total number of platelets. Optionally the combined plasma supply is centrifuged at 3000 rpm for approximately 25 minutes, and the plasma is optionally transferred to another sterile container.

According to at least one embodiment, the pellet is re-suspended with sufficient plasma to make the final platelet concentration approximately 1×10⁹ per mL, an amount referred to as 100% concentration. Optionally, thereafter the plasma/platelet suspension is frozen and allowed to thaw to lyse the platelets. According to one embodiment, the suspension is chilled to approximately negative 20° C. overnight and allowed to thaw at about 37° C. for about 10 minutes. This optional step may be repeated multiple times, for example, between 2 to 20 times to ensure lysing. Thereafter, the resulting hPL is centrifuged to remove the platelet debris, which may occur at about 3000 rpm for about 10 min. Thereafter, the resulting hPL is optionally decanted into a sterile bottle, and is then optionally filtered with an approximately 0.22 μm filter.

According to certain embodiments, the resulting solution is screened for infectious agents. Alternatively, the solution may be utilized directly as a non-animal-derived tissue culture supplement. However, according to certain embodiments, CaCl₂ may be added to the solution as a supplement to the resulting hPL medium. For example,

### EXAMPLES

As no instances of utilizing CB hPL solutions for in vitro culture of therapeutic cell types have been reported, tests were conducted to determine whether CB hPL could support the in vitro culture of therapeutic cell types, such as MSCs. Further, tests were conducted to determine how such a solution would compare with FBS, and whether CB derived hPL solutions could support such cultures at reduced concentrations to make a suitable substitute for FBS for clinical cell manufacturing. Initial experiments were performed using mesenchymal stem/stromal cells (MSC) from various tissue sources. Primary MSC cultures which were previously established using FBS and then cryopreserved were utilized for the following tests. Those cultures were then thawed and subcultured using medium containing either FBS or CB hPL. Culture experiments were performed in parallel using identical cryopreserved MSC cultures to test the primary variable of FBS vs. CB hPL. The following parameters were studied:
1. Growth rate/doubling time to determine proliferative capacity;
2. Colony forming units-fibroblast (CFU-F) to determine stem/progenitor cell potential;
3. Differentiation into MSC lineages (such as osteogenic and adipogenic); and
4. Expression/retention of surface markers characteristic of MSC.

The results from those tests indicated that in testing each of the parameters, a CB hPL solution produced according to at least one embodiment herein was found to perform substantially similarly to FBS. Specifically, the growth rate/doubling time utilizing a 100% solution was found to be similar to that of FBS for those cells tested. Further, the solution was found to have similar stem/progenitor cell potential to FBS. Additionally, those cells cultured in the solution were found to differentiate into various MSC lineages similar to FBS, and there were no apparent issues with the expression or retention of surface markers that are characteristic of the tested MSCs.

### Example 1

### Comparison of CB hPL Composition to FBS

Turning now to FIGS. 1A and 1B, multiple samples of hPL culture media were made according to different protocols. Specifically, sample hPL4 was prepared by utilizing platelets concentrated using Ficoll density gradient from a 3 day old CB unit. According to another embodiment, sample hPL5 was prepared by utilizing platelets collected by centrifugation from a 3 day old CB unit. Further, sample hPL6, was prepared from CB plasma greater than 2 weeks old that was leftover from cell isolation. According to certain embodiments, platelets were collected by centrifugation. After culturing placental MCSs within these solutions for 24 hours and 96 hours, the cultures were inspected, with cell counts made. FIGS. 1A and 1B are images of the resulting cultures. As can be seen, in a short-term culture assay, each of the CB hPL compositions support MSC growth and morphology with similar, if not better, results than utilizing FBS. Ficoll concentration solutions of platelets resulted in similar results to other CB hPL compositions. Further, the addition of CaCl₂ to medium containing CB hPL further results in an effective solution MSC proliferation composition.

### Example 2

### Appearance and Morphology of MSC

According to certain embodiments, three different media were prepared for comparison of MSC development and morphology. In one exemplary embodiment, Medium A=DMEM 45 mL+FBS 5 mL; Medium B=DMEM 45 mL+hPL 5 mL+2 units/mL of heparin; and Medium C=DMEM 45 mL+hPL 5 mL+2 mM CaCl2+2 units/mL of heparin. Cryopreserved placental MSC were thawed in medium containing FBS. Thereafter, those thawed placental MSCs were split into cultures with equal number of cells on each of the three media. Growth and gross morphology were observed after 24 hours and after 5 days in culture. As shown in FIG. 2, CB hPL compositions supported MSC growth and morphology at least as well as FBS, and CB hPL compositions including CaCl₂ performed better than FBS. Additional tests were performed culturing dental papillary stem cells, resulting in similar results.

### Example 3

### Varying hPL Composition Dilution

According to certain embodiments, dental papillary stem cells (“DPSC”) and mesenchymal stem cells (“MSC”) were cultured on varying concentrations of hPL compositions made according to methods disclosed herein, with colony doubling time being recorded. As discussed above, 100% concentration of platelets in these tests is considered to be 1×10⁹ platelets/ml of plasma. Therefore, the hPL obtained by lysing 1×10⁹ platelets/ml of plasma was considered as standard a hPL or 100% hPL.

In order to test the effect of hPL dilution on cell growth, two separate hPL media were prepared by diluting hPL compositions to 50% and 25% using the left over plasma. Therefore, the 50% hPL contained 0.5×10⁹ platelets/ml of plasma and 25% hPL contained 0.25×10⁹ platelets/ml of plasma. These tests were performed on two different occasions, with the first results being reported in FIG. 3A and the second set of test being reported in FIG. 3B. It will be appreciated that the first test results, shown in FIG. 3A do not show a statistically significant decrease in doubling time, even when the hPL composition concentration is reduced to 50% and 25%. Further, according to the second set of test results shown in FIG. 3B, the reduction in hPL concentration produced DTs that were statistically higher that the full concentration composition, but not at a rate that is as high as the dilution. For instance, a closer look at the data from test two shows the surprising result that lowering hPL concentration to ½ or ¼th did not result in corresponding 2-fold and 4-fold increase in DT, respectively. Taking these two tests together, there is an indication that reducing hPL concentration may or may not reduce the growth rate of cells, but that reduction in concentration, at worst does not linearly effect the growth rate. This is significantly different from results with FBS. Further, the above tests with reduced concentrations of hPL compositions showed that both MSCs and DPSCs grew with healthy morphology and reached confluence even at the greatly reduced concentration, as shown in FIG. 3C.

### Example 4

### Comparing Doubling Time on Stem Cells

According to certain exemplary embodiments, DPSC, placental MSC and cord matrix MSC previously established using FBS were thawed and cultured in different media to compare the doubling time and morphology for the following compositions. As shown in FIG. 4A, placental MSCs were cultured on FBS, hPL4, hPL5, hPL6, and hPL6 plus CaCl₂ compositions as discussed above, with doubling times recorded as shown. In this test, hPL4 platelets were concentrated using Ficoll density gradient from a 3 day old CB unit; the hPL5 platelets were collected by centrifugation from a 3 day old CB unit; and the hPL6, CB plasma (>2 weeks old) was leftover from cell isolation. Platelets were collected by centrifugation. As can be seen, each of these compositions had a higher doubling time than FBS, although the addition of CaCl₂ in the final composition resulted in a similar doubling time to FBS.

By comparison, turning now to FIG. 4D, the experimental results from using adult hPL without CaCl₂ resulted in a drastically higher doubling time of over 200 hours, as compared to the CB hPL examples shown in FIG. 4A. It will be appreciated that addition of CaCl₂ to the adult hPL resulted in significant reduction in doubling time, similar to those seen in FBS and CB hPL.

Turning now to FIGS. 4B and 4C, two separate tests were performed to compare the doubling time of DPSCs when cultured on a CB hPL composition according to at least one embodiment herein versus FBS under identical conditions. As can be seen, both CB hPL compositions (one including CaCl₂) resulted in a doubling time similar to FBS. Finally, turning to FIG. 4F, the doubling time of cord MSCs when cultured on CB hPL composition according to at least one embodiment herein was compared to FBS under identical conditions. As can be seen, the doubling time of both compositions were comparable.

### Example 5

### Effect on Differentiation of Stem Cells

According to at least one exemplary embodiment, human DPSCs and human cord MSCs were cultured in appropriate differentiation medium for 3 weeks and then stained for tissue-specific differentiation as has been previously described by Woods et al., Cryobiology 59 (2) 150-157 (October 2009), the contents of which are incorporated herein by reference. According to observation over the culture period, CB hPL supplemented with CaCl₂ supported osteogenic and adipogenic differentiation at least as well as FBS.

### Example 6

Maintenance of MSC phenotype in Culture with FBS vs. CB hPL

According to at least one exemplary embodiment, human DPSCs and human cord MSCs were cultured for two weeks in medium containing either FBS or CB hPL. MSCs were analyzed by flow cytometry for expression of a limited panel of MSC surface markers using previously described methods disclosed by Woods et al., Cryobiology 59 (2) 150-157 (October 2009), the contents of which are incorporated herein by reference. Antigen expression was determined relative to isotype control antibody, and was determined for a minimum of three independent MSC cultures. These tests found that CD73 expression was greater than or equal to 95% positive; CD90 expression was greater than or equal to 95% positive; CD146 expression was greater than or equal to 95% positive; HLA-DR expression was less than than or equal to ≦5% positive; and CD45 expression was less than or equal to 5% positive, indicating that CB hPL compositions according to at least one embodiment herein did not interfere with the expression of surface marker production, resulting in a phenotype similar to cultures grown on FBS.

What is claimed is:

1. A composition for culturing therapeutic cells, the composition comprising human platelet lysate and plasma derived from cord blood, wherein the concentration of platelet lysate is approximately the equivalent of 1×10⁹ platelets/ml of composition, and wherein the plasma and/or platelets are operable to culture one or more therapeutic cells.

2. The composition of claim 1, further comprising approximately 0.5 mM/mL to about 5 mM/mL calcium chloride.

3. The composition of claim 1, wherein the concentration of platelet lysate is approximately the equivalent of 0.5×10⁹ platelets/ml of composition.

4. The composition of claim 1, wherein the concentration of platelet lysate is approximately the equivalent of 0.5×10⁹ platelets/ml of composition.

5. The composition of claim 3, wherein the composition is operable to proliferate human mesenchymal stem cells or dental papillary stem cells.

6. The composition of claim 3, wherein the composition is operable to proliferate human stem cells at a rate less than double that of fetal bovine serum under identical conditions.

7. The composition of claim 4, wherein the composition is operable to proliferate human stem cells at a rate less than four times that of fetal bovine serum under identical conditions.

8. The composition of claim 2, further comprising heparin.

9. A method for culturing therapeutic cells comprising the steps of:
a. providing a therapeutic cell;
b. providing a composition derived from cord blood consisting essentially of combined human platelet lysate and plasma, wherein the concentration of platelet lysate is approximately the equivalent of 1×10⁹ platelets/ml of composition;
c. culturing the therapeutic cell on the composition.

10. The method of claim 9, wherein the composition further comprises approximately 0.5 mM/mL to about 5 mM/mL calcium chloride.

11. The method of claim 9, wherein the concentration of platelet lysate is approximately the equivalent of 0.5×109 platelets/ml of composition.

12. The method of claim 9, wherein the concentration of platelet lysate is approximately the equivalent of 0.5×109 platelets/ml of composition.

13. The method of claim 9, wherein the therapeutic cell is mesenchymal stem cells or dental papillary stem cells.

14. The method of claim 13, wherein the therapeutic cell is mesenchymal stem cells or dental papillary stem cells.

15. The method of claim 9, further comprising the step of diluting the platelet lysate with additional plasma such that the resulting concentration is between about half to about one quarter of the original concentration.

16. The method of claim 15, wherein the step of culturing the therapeutic cell on the composition occurs at a rate of less than four times that of a therapeutic cell cultured at the original concentration.

17. A composition for culturing therapeutic cells, the composition consisting essentially of human platelet lysate and plasma derived from cord blood, wherein the concentration of platelet lysate is approximately the equivalent of 1×10⁹ platelets/ml of composition, and wherein the plasma and/or platelets are operable to culture one or more therapeutic cells.

18. The composition of claim 18, further consisting of calcium chloride.

19. The composition of claim 19, further consisting of heparin.
